(19) United States
(12) Patent Application Publication
(10) Pub. No.: US 2007/0116783 A1
(43) Pub. Date: May 24, 2007

(54) Silver-based inorganic antibacterial agent dispersion

(76) Inventor: Yasuharu Ono, Aichi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/587,208

(22) PCT Filed: Jan. 26, 2004
(86) PCT No.: PCT/JP04/00638

§ 371 Date: Jul. 25, 2006

(51) Int. Cl. A61K 33/38

(52) U.S. Cl. 424/618

(57) ABSTRACT

The invention provides a silver-based inorganic antibacterial agent dispersion that has excellent dispersibility and storage stability and allows the silver-based inorganic antibacterial agent to fully exhibit its antibacterial performance. Furthermore, it provides an antibacterial-processed product that is processed using this silver-based inorganic antibacterial agent dispersion. The silver-based inorganic antibacterial agent dispersion of the present invention is one that includes a silver-based inorganic antibacterial agent, a discoloration inhibitor, a thickener, a dispersant, and a dispersion medium. It is one that includes 5 to 60 parts by mass of the silver-based inorganic antibacterial agent in 100 parts by mass of the above dispersion, and 0.1 to 10 parts by mass of the thickener relative to 100 parts by mass of the silver-based inorganic antibacterial agent, one that includes an imidazole series compound and/or a benzotriazole series compound as the discoloration inhibitor, and one that includes a polysaccharide or a cellulose-based thickener as the thickener.

### TECHNICAL FIELD

The invention relates to a silver-based inorganic antibacterial agent dispersion having excellent dispersibility and storage stability, and to an antibacterial-processed product that is processed using the silver-based inorganic antibacterial agent dispersion.

### BACKGROUND ART

It has been known for a long time that silver ion has antibacterial and antimold actions. From this knowledge, various silver-based inorganic antibacterial agents having silver ion supported on various types of inorganic materials have been proposed. Since the silver-based inorganic antibacterial agents have higher safety than organic antibacterial agents and do not vaporize or decompose, the antibacterial effects are sustained for a long period of time, and their heat resistance is excellent. Due to such properties, an antibacterial resin composition formed from a mixture of a silver-based inorganic antibacterial agent and various types of macromolecular compound can be processed into fiber, film, various types of moldings, etc. These processed products, etc. are used in various types of applications where antibacterial action is required.

The silver-based inorganic antibacterial agent may be attached to fiber, nonwoven fabric, a filter, or film by dipping or coating using a solution in which it is dispersed with a binder, a solvent, etc. or may be used when wet-spinning fiber. In such a case, it is necessary to disperse the silver-based inorganic antibacterial agent in water, a solvent, etc. However, since the silver-based inorganic antibacterial agent aggregates in a dispersion, processing of a product might become poor or the antibacterial performance of the processed products might vary.

In order to solve these problems, a method has been proposed in which a, silver-based inorganic antibacterial agent dispersion is prepared in advance by uniformly dispersing a silver-based inorganic antibacterial agent in water, a solvent, a paint, etc. at high concentration, and diluting this as appropriate with water, a solvent, a paint, etc. Ref., for example, JP-A-06-263612 and JP-A-11-104218 (JP-A denotes a Japanese unexamined patent application publication).

Furthermore, as described in the above-mentioned laid-open patent applications, using a dispersant such as a surfactant in combination when adding a silver-based inorganic antibacterial agent to water, a solvent, a paint, etc. is a general technique. However, even if the dispersibility of the silver-based inorganic antibacterial agent is improved by the addition of a dispersant, there is still the problem that, when this dispersion is stored, the silver-based inorganic antibacterial agent precipitates, the precipitated silver-based inorganic antibacterial agent further coagulates, and it becomes difficult to disperse again. Moreover, many dispersants react with silver ion, and when the silver-based inorganic antibacterial agent is used in combination with a dispersant, there is the problem that the silver-based inorganic antibacterial agent and the dispersant react with each other, thus causing discoloration or degrading the antibacterial performance.

### DISCLOSURE OF THE INVENTION

The present invention provides a silver-based inorganic antibacterial agent dispersion that has excellent dispersibility and storage stability and allows the silver-based inorganic antibacterial agent to fully exhibit its antibacterial performance. Furthermore, it provides an antibacterial-processed product that is processed using this silver-based inorganic antibacterial agent dispersion.

As a result of an intensive investigation by the present inventors in order to solve the above-mentioned problems, it has been found that they can be solved by a silver-based inorganic antibacterial agent dispersion comprising a silver-based inorganic antibacterial agent, a discoloration inhibitor, a thickener, a dispersant, and a dispersion medium. It has also been found that dispersibility and storage stability are excellent when 100 parts by mass of the dispersion contains 5 to 60 parts by mass of the silver-based inorganic antibacterial agent, and there is 0.1 to 10 parts by mass of the thickener relative to 100 parts by mass of the silver-based inorganic antibacterial agent, and the present invention has thus been accomplished. Furthermore, it has been found that by the use of an imidazole series compound and/or a benzotriazole series compound as the discoloration inhibitor a silver-based inorganic antibacterial agent dispersion having excellent discoloration resistance can be obtained, and that by the use of a polysaccharide or a cellulose-based thickener as the thickener a silver-based inorganic antibacterial agent dispersion having very good storage stability can be obtained, and the present invention has thus been accomplished. Moreover, antibacterial-processed products processed using the silver-based inorganic antibacterial agent dispersion of the present invention have hardly any processing failures, and excellent antibacterial performance can be exhibited.

The present invention has been accomplished based on the above-mentioned knowledge, and representative examples thereof are illustrated below.
1. A silver-based inorganic antibacterial agent dispersion comprising a silver-based inorganic antibacterial agent, a discoloration inhibitor, a thickener, a dispersant, and a dispersion medium.
2. The silver-based inorganic antibacterial agent dispersion according to 1 above, wherein the silver-based inorganic antibacterial agent is 5 to 60 parts by mass relative to 100 parts by mass of the silver-based inorganic antibacterial agent dispersion, and the thickener is 0.1 to 10 parts by mass relative to 100 parts by mass of the silver-based inorganic antibacterial agent.
3. A silver-based inorganic antibacterial agent dispersion formed by further adding a fine particulate compound having an average particle size of 1 to 100 nm to the silver-based inorganic antibacterial agent dispersion according to 1 above.
4. A silver-based inorganic antibacterial agent dispersion formed by further adding a fine particulate compound having an average particle size of 1 to 100 nm to the silver-based inorganic antibacterial agent dispersion according to 2 above.
5. The silver-based inorganic antibacterial agent dispersion according to any one of 1 to 4 above, wherein the discoloration inhibitor is an imidazole series compound and/or a benzotriazole series compound.
6. The silver-based inorganic antibacterial agent dispersion according to any one of 1 to 4 above, wherein the thickener is a polysaccharide and/or a cellulose-based thickener.
7. The silver-based inorganic antibacterial agent dispersion according to 6 above, the thickener is a polysaccharide. 8. The silver-based inorganic antibacterial agent dispersion according to any one of 1 to 4 above, wherein the dispersant is an anionic surfactant and/or a nonionic surfactant.
9. The silver-based inorganic antibacterial agent dispersion according to 8 above, wherein the dispersant is an anionic surfactant.
10. The silver-based inorganic antibacterial agent dispersion according to any one of 1 to 4 above, wherein it further comprises a binder resin.
11. An antibacterial-processed product processed using the silver-based inorganic antibacterial agent dispersion according to any one of 1 to 4 above.

### BEST MODE FOR CARRYING OUT THE INVENTION

The present invention is explained in detail below.

Silver-Based Inorganic Antibacterial Agent

A silver-based inorganic antibacterial agent used in the present invention is an inorganic compound containing silver ion, and preferably has a maximum particle size of substantially no greater than 10 μm, and more preferably a maximum particle size of substantially no greater than 5 μm. A silver-based inorganic antibacterial agent dispersion in which particles of the silver-based inorganic antibacterial agent having a maximum particle size exceeding 10 μm are present might form a precipitate or might not be dispersed uniformly in a paint, etc., and the surface smoothness of an antibacterial-processed product processed using same might be impaired. ‘Substantially’ referred to here means that the weight of a group of particles with the maximum particle size or less is at least 98% of the total weight of the particles, preferably at least 99%, and more preferably at least 99.5%.

The average particle size of the silver-based inorganic antibacterial agent is not particularly limited, but it is preferably 0.1 to 5 μm, more preferably 0.35 to 4 μm, and particularly preferably 0.5 to 2 μm. When the average particle size is less than 0.1 μm, the silver-based inorganic antibacterial agent easily aggregates and might become difficult to handle. When the average particle size exceeds 5 μm, the silver-based inorganic antibacterial agent particles easily precipitate and handling might become difficult.

Specific examples of the silver-based inorganic antibacterial agent include antibacterial zeolites, antibacterial amorphous aluminosilicates, antibacterial intercalation compounds, antibacterial soluble glasses, and antibacterial phosphate salts.

The antibacterial zeolite is a zeolite, etc. in which an ion-exchangeable ion in the zeolite is substituted by a silver ion, such as those described in JP-B-63-54013 (JP-B denotes a Japanese examined patent application publication), JP-A-60-181002, JP-A-63-265809, JP-A-2-111709, JP-A-3-145410, etc.

The antibacterial amorphous aluminosilicate is an amorphous aluminosilicate, etc. in which an ion-exchangeable ion is substituted by a silver ion, such as those described in JP-A-62-70221, JP-A-1-167212, etc.

The antibacterial intercalation compound is, for example, one described in JP-A-1-2213034, or an intercalation compound in which a silver compound is supported on an inorganic layered compound.

The antibacterial soluble glass is an antibacterial soluble glass containing silver ion, such as those described in JP-A-62-158202, JP-A-62-21098, JP-A-63-48366, JP-A-1-213410, etc.

The antibacterial phosphate salt is a silver ion-containing phosphate salt series compound, etc., such as those described in JP-A-1-221304, JP-A-3-83905, etc.

Among these silver-based inorganic antibacterial agents, the antibacterial zeolite and the antibacterial phosphate salt can preferably be used in the present invention since it is easy to control the particle size and, in particular, the antibacterial phosphate salt can more preferably be used since its discoloration resistance is excellent.

In 100 parts by mass of the silver-based inorganic antibacterial agent dispersion of the present invention, the silver-based inorganic antibacterial agent is 5 to 60 parts by mass, preferably 7 to 55 parts by mass, more preferably 10 to 50 parts by mass, and particularly preferably 12 to 45 parts by mass. If the silver-based inorganic antibacterial agent is less than 5 parts by mass relative to 100 parts by mass of the silver-based inorganic antibacterial agent dispersion of the present invention, the silver-based inorganic antibacterial agent easily separates from the dispersion and the storage stability might be degraded. In this case, exhibition of the antibacterial effect might become unstable. If the silver-based inorganic antibacterial agent exceeds 60 parts by mass relative to 100 parts by mass of the silver-based inorganic antibacterial agent dispersion of the present invention, the viscosity of the dispersion might become too high, thus making it difficult to produce and degrading the ease of handling of the product. mass relative to 100 parts by mass of the silver-based inorganic antibacterial agent, preferably 0.6 to 7 parts by mass, and particularly preferably 1 to 5 parts by mass. If the mixing ratio of the thickener is less than 0.1 parts by mass relative to 100 parts by mass of the silver-based inorganic antibacterial agent, the precipitation prevention effect of the antibacterial agent might be small, and if it exceeds 10 parts by mass, the viscosity of the dispersion might become too high, thus making it difficult to produce or degrading the ease of handling of the product.

Dispersant

A dispersant used in the silver-based inorganic antibacterial agent dispersion of the present invention is not particularly limited, and examples thereof include anionic surfactants such as an alkenylsuccinic acid salt, an alkylbenzene sulfonic acid salt, an alkylnaphthalene sulfonic acid salt, an alkylsulfate ester, a fatty alcohol sulfate ester, a polyoxyethylene alkylether sulfate ester, a dialkylsulfosuccinate salt, phosphate ester series such as an alkylphosphate ester and a phosphate ester series copolymer, and a polycarboxylic acid type macromolecular surfactant; nonionic surfactants such as a polyoxyethylene alkyl ether, a polyoxyethylene alkyl allyl ether, and an organic modified organopolysiloxane; cationic surfactants such as an alkylamine salt and a quaternary ammonium salt; betaine type amphoteric surfactants such as an alkylbetaine and an amidobetaine; polyphosphoric acid salts such as a pyrophosphoric acid salt and a tripolyphosphoric acid salt; and polyamines such as triethanolamine. As the dispersant used in the present invention, the anionic surfactant is preferable, the phosphate ester series or the polycarboxylic acid type macromolecular surfactant are more preferable, and the phosphate ester series is particularly preferable. In this phosphate ester series, those in which the basic skeleton is formed from an ester chain, a vinyl chain, an acrylic chain, an ether chain, a urethane chain, etc, are preferable, and some of the hydrogen atoms in the molecule may be substituted by a halogen atom. Among these, an acrylic resin, a polyester resin, an alkyd resin, etc. are preferable, and an acrylic resin or a polyester resin is particularly suitable. The dispersant may employ a plurality thereof in combination.

The mixing ratio of the dispersant of the silver-based inorganic antibacterial agent dispersion of the present invention is preferably 0.1 to 15 parts by mass relative to 100 parts by mass of the silver-based inorganic antibacterial agent, more preferably 1 to 12 parts by mass, and particularly preferably 2 to 10 parts by mass. If the mixing ratio is less than 0.1 parts by mass relative to 100 parts by mass of the silver-based inorganic antibacterial agent, the dispersion effect becomes inadequate and aggregation might easily occur. If it exceeds 15 parts by mass, the excess amount of dispersant might badly affect and degrade the dispersibility, and the antibacterial action might be degraded.

Dispersion Medium

A dispersion medium in the silver-based inorganic antibacterial agent dispersion of the present invention is preferably water or a water-soluble solvent. Specific examples of the dispersion medium include water, alcohols such as ethanol and isopropanol, dimethylformamide, dimethylacetamide, dimethylsulfoxide, tetrahydrofuran, and acetone. The dispersion medium may employ a plurality of solvents as a mixture.

The dispersion medium used in the present invention may be used as a solvent for diluting the silver-based inorganic antibacterial agent dispersion of the present invention or as a solvent for mixing with another substance.

The dispersion medium may be selected as appropriate according to the type of paint, etc. or the physical properties of an antibacterial-processed product processed using the present dispersion.

The dispersion medium of the silver-based inorganic antibacterial agent dispersion of the present invention may be added so that, together with

Discoloration Inhibitor

A discoloration inhibitor used in the silver-based inorganic antibacterial agent dispersion of the present invention prevents discoloration when storing or processing the dispersion. It also prevents discoloration of an antibacterial-processed product, processed using the silver-based inorganic antibacterial agent dispersion of the present invention, due to the silver-based inorganic antibacterial agent.

The discoloration inhibitor used in the silver-based inorganic antibacterial agent dispersion of the present invention is not particularly limited as long as it has an effect in preventing discoloration due to silver. For example, the discoloration inhibitor and silver or silver ion bond to form a colorless and/or white compound, and the discoloration inhibitor is preferably stable in the presence of an acid or an alkali; specific examples thereof include the compounds below. That is, examples of the discoloration inhibitor include benzotriazole series compounds such as methylbenzotriazole and a potassium salt of methylbenzotriazole; triazole series compound such as α-[2-(4-chlorophenyl)ethyl]-α-(1,1-dimethylethyl)-1H-1,2,4-triazol-1-yl-ethanol; imidazole series compounds such as imidazole, benzimidazole, and 2-methylimidazole; cyanuric acid series compounds such as cyanuric acid and isocyanuric acid; triazine series compounds such as melamine; ammonium salts such as ammonium polyphosphate; nitrogen-containing compounds such as polyphosphoric amide; oxalic acid anilide series compounds; salicylic acid series compounds; hindered amine series compounds; and hindered phenol series compounds. These compounds may be used in a combination of two or more types.

As the discoloration inhibitor used in the present invention, a benzotriazole series compound and/or an imidazole series compound are preferable, and it is particularly preferable to use a benzotriazole series compound and an imidazole series compound in combination.

The mixing ratio of the discoloration inhibitor in the silver-based inorganic antibacterial agent dispersion of the present invention is 0.5 to 20 parts by mass relative to 100 parts by mass of the silver-based inorganic antibacterial agent, preferably 1 to 15 parts by mass, and more preferably 2 to 10 parts by mass. If the mixing ratio of the discoloration inhibitor is less than 0.5 parts by mass relative to 100 parts by mass of the silver-based inorganic antibacterial agent, the discoloration inhibition effect might not be fully exhibited, and if it exceeds 20 parts by mass, the excess amount of discoloration inhibitor might badly affect the antibacterial action or the physical properties of the dispersion.

Thickener

A thickener used in the silver-based inorganic antibacterial agent dispersion of the present invention prevents precipitation or aggregation of the silver-based inorganic antibacterial agent during storage of the silver-based inorganic antibacterial agent dispersion, and prevents precipitated silver-based inorganic antibacterial agent from aggregating or solidifying to thus degrade the dispersibility of the silver-based inorganic antibacterial agent dispersion.

Specific examples of the thickener include cellulose-based thickeners such as methyl cellulose, carboxymethyl cellulose, methyl hydroxy cellulose, methyl hydroxypropyl cellulose, and hydroxyethyl cellulose; polysaccharides such as xanthan gum, gum arabic, tragacanth gum, guar gum, tamarind gum, and carrageenan; various types of polyacrylamide series polymers; polyethylene oxide; polyethylene glycol; polyvinyl alcohol; and clay. The thickener may employ a plurality thereof in combination.

Among these thickeners, the cellulose-based thickener and/or the polysaccharide are preferable, the polysaccharide is more preferable, and xanthan gum is particularly preferable.

The mixing ratio of the thickener in the silver-based inorganic antibacterial agent dispersion of the present invention is 0.1 to 10 parts by the silver-based inorganic antibacterial agent, the discoloration inhibitor, the thickener, and the dispersant, the total of the silver-based inorganic antibacterial agent dispersion becomes 100 parts by mass.

Binder Resin

The silver-based inorganic antibacterial agent dispersion of the present invention may be used with an acrylic acid-based binder resin, a urethane-based binder resin, etc., which are normally used for the surface treatment of fiber, nonwoven fabric, a sheet, etc. The binder resin used here may employ a plurality thereof as a mixture.

The mixing ratio of the binder resin is preferably 10 to 300 parts by mass relative to 100 parts by mass of the silver-based inorganic antibacterial agent in the silver-based inorganic antibacterial agent dispersion, and more preferably 20 to 250 parts by mass. If the binder resin is less than 10 parts by mass relative to 100 parts by mass of the silver-based inorganic antibacterial agent, when the antibacterial agent is attached to fiber, nonwoven fabric, a sheet, etc., the fixation strength might not be sufficient, the silver-based inorganic antibacterial agent might come off, and the antibacterial performance might be degraded. If the binder resin exceeds 300 parts by mass relative to 100 parts by mass of the silver-based inorganic antibacterial agent, the storage stability of the silver-based inorganic antibacterial agent dispersion might be degraded, and when it is processed into fiber, nonwoven fabric, a sheet, etc., the silver-based inorganic antibacterial agent might be covered with the binder resin, and the antibacterial performance might not be exhibited fully.

Process for Producing Silver-Based Inorganic Antibacterial Agent Dispersion

Production of the silver-based inorganic antibacterial agent dispersion of the present invention may be carried out by any standard method as long as it is for preparing a dispersion of an inorganic powder. For example, the silver-based inorganic antibacterial agent, the discoloration inhibitor, the thickener, the dispersant, and the dispersion medium may be dispersed by stirring and mixing by means of a sand mill, a disper, a ball mill, etc. The order of mixing the silver-based inorganic antibacterial agent, etc. is not limited, but it is preferable that material that is difficult to disperse or dissolve in the dispersion medium is dispersed in advance in a small amount of solvent or dissolved in a solvent in which it is soluble, and then mixed. A treatment for removing foreign material or aggregates after the dispersion may be carried out. Examples of such a treatment include passing through a fine mesh (sieving).

If desired, the silver-based inorganic antibacterial agent dispersion may contain an antifoaming agent, a preservative, an antimold agent, a corrosion inhibitor, an ultraviolet absorber, an antioxidant, a fluorescent agent, a metal powder, a filler, a colorant such as a pigment or a dye, a flame retardant, a deodorant, an inorganic antibacterial agent or organic antibacterial agent other than the silver-based antibacterial agent, and/or a softener, etc. For example, there are antifoaming agents having foam breaking properties, foam suppressing properties, or foam removing properties, and any type may be used. Examples of the foam-breaking antifoaming agent include a polysiloxane solution and a mixture of a non-silicone type foam-breaking polymer and hydrohobic particles.

When the silver-based inorganic antibacterial agent dispersion of the present invention is used for fiber, nonwoven fabric, a sheet, etc., the original color of the substrate might be impaired. For example, if the silver-based inorganic antibacterial agent dispersion is used for a black fiber or textile, it might become partly white. In this case, by adding a fine particulate compound having an average particle size of 1 to 100 nm to the silver-based inorganic antibacterial agent dispersion of the present invention, such a change in color can be suppressed.

Fine Particulate Compound

The fine particulate compound added to the silver-based inorganic antibacterial agent dispersion of the present invention has an average particle size of 1 to 100 nm, preferably 5 to 50 nm, and more preferably 10 to 40 nm. This fine particulate compound is preferably a colloidal compound. The colloidal compound is formed from colloid particles of a single inorganic oxide or formed from a composite oxide, a hydroxide, or a mixture thereof, and any known colloidal compound may be used.

Specific examples of the fine particulate compound added to the silver-based inorganic antibacterial agent dispersion of the present invention include single inorganic oxide colloid particles and composite oxide colloid particles. Examples of the single inorganic oxide colloid particles include Al₂O₃, SiO₂, TiO₂, ZrO₂, and ZnO₂. Examples of the composite oxide colloid particles include SiO₂.Al₂O₃, SnO₂·Sb₂O₃, TiO₂.ZrO₂.SiO₂, SiO₂.TiO₂.Al₂O₃, SiO₂.Al₂O₃.MgO, and SiO₂.Al₂O₃.CaO. Among them, single oxide colloid particles of Al₂O₃ or SiO₂ are particularly preferable, and single inorganic oxide colloid particles of SiO₂, that is, the so-called colloidal silica, are most preferable. Examples thereof include silica sol (ST-C manufactured by Nissan Chemical Industries Ltd., SiO₂ concentration about 20 wt %) and alumina sol (alumina sol 520 manufactured by Nissan Chemical Industries Ltd., Al₂O₃ concentration about 25%). The fine particulate compound may employ a plurality thereof as a mixture.

The amount of fine particulate compound added to the silver-based inorganic antibacterial agent dispersion of the present invention is 10 to 500 parts by mass relative to 100 parts by mass of the silver-based inorganic antibacterial agent, preferably 20 to 400 parts by mass, and more preferably 30 to 300 parts by mass.

Fiber

Fiber and textile to which the silver-based inorganic antibacterial agent dispersion containing the fine particulate compound is applied are not particularly limited, and all known fibers and textiles may be employed. Examples thereof include cotton, acrylic, polyester, polyurethane, and nylon, and blended spun products of these fibers may also be employed.

With regard to the fiber or textile to which the silver-based inorganic antibacterial agent dispersion containing the fine particulate compound is applied, it is preferable that the difference between an L value, measured using a colorimeter, of the silver-based inorganic antibacterial agent and an L value of one that the dispersion is applied to is at least 10, more preferably at least 20, and particularly preferably at least 30. The difference in L value is at most 90, and preferably at most 85. If the difference in L value is less than 10, no difference in color might be seen between the fiber or textile obtained using the silver-based inorganic antibacterial agent dispersion containing the fine particulate compound and the fiber or textile obtained using the silver-based inorganic antibacterial agent dispersion alone. That is, a fiber with good color can easily be obtained by applying the silver-based inorganic antibacterial agent dispersion containing no fine particulate compound. The color difference is in accordance with JIS Z8729.

Method of Adhering to Fiber

As a method of adhering the silver-based inorganic antibacterial agent dispersion containing the fine particulate compound to fiber or textile, a known method may be employed. For example, as an adhering method employing same, fiber or textile is immersed in the dispersion, then squeezed, and dried by means of an air dryer, etc. As another adhering method, fiber or textile is sprayed with the dispersion and then dried. In this case, the dispersion may contain a binder resin, and as the binder resin any known binder resin may be used. Examples of the binder resin include acrylic or urethane-based resins. The amount of binder resin adhered may be set according to the intended application. The dispersion may contain a softener, etc. in order to improve the feel of the fiber or textile after the dispersion is adhered thereto.

Use

The silver-based inorganic antibacterial agent dispersion of the present invention may be applied to various products where antibacterial action is required. For example, fiber, nonwoven fabric, a sheet, etc. may be immersed in a process solution formed by diluting the dispersion of the present invention with water or an aqueous emulsion, etc. to thus make the antibacterial agent become attached thereto. For fiber produced by a wet spinning method, such as acrylic fiber, the dispersion of the present invention may be added to a spinning solution or a solvent to thus process it into fiber with the antibacterial agent incorporated therein. Furthermore, by mixing a water-based paint with the dispersion of the present invention, a paint having antibacterial action may be obtained. By applying this paint to a substrate, antibacterial action can simply be imparted to various types of materials. Furthermore, the dispersion of the present invention may be used as a disinfectant or a disinfectant spray.

The amount of dispersion of the present invention added or the dilution ratio thereof may be selected as appropriate depending on the required performance. For example, when processing a fiber product or a paint using the dispersion of the present invention, the dispersion may be prepared so that 0.01 to 1 g/m² of the silver-based inorganic antibacterial agent adheres to the processed product. When the silver-based inorganic antibacterial agent is incorporated into a resin as in wet spinning or a urethane sponge, etc., it may be added and/or diluted so that the dispersion of the present invention is 0.1 to 5 parts by mass relative to 100 parts by mass of the resin solids content.

Specific examples of applications include various types of fiber such as underwear, stockings, shirts, socks, duvets, duvet covers, cushions, blankets, carpets, curtains, couches, car seats, air filters, and wall paper, nonwoven fabric, paper products, sponges, paints, and floor wax.

### EMBODIMENT

A silver-based inorganic antibacterial agent dispersion comprising a silver-based inorganic antibacterial agent, a discoloration inhibitor, a thickener, a dispersant, and a dispersion medium, wherein the silver-based inorganic antibacterial agent is 5 to 60 parts by mass relative to 100 parts by mass of the silver-based inorganic antibacterial agent dispersion, and the thickener is 0.1 to 10 parts by mass relative to 100 parts by mass of the silver-based inorganic antibacterial agent.

### EXAMPLES

The present invention is specifically explained below, but the present invention is not limited thereby.

A preparation method for the silver-based inorganic antibacterial agent dispersion, various evaluation test methods for samples obtained, and results thereof are as follows.

Preparation of Silver-Based Inorganic Antibacterial Agent Dispersion

Compounds used in Examples and Comparative Examples are described below.
Antibacterial agent: silver-supporting zirconium phosphate (product name
Novaron AG300, manufactured by Toagosei Co., Ltd.)
Discoloration inhibitor (A): imidazole
Discoloration inhibitor (B): methylbenzotriazole
Dispersant: BYK-180 (product name, alkylammonium salt of phosphate group-containing block copolymer manufactured by Byk-Chemie (Germany))
Thickener (C): xanthan gum (product name Eko gum T, manufactured by Dainippon Pharmaceutical Co., Ltd.)
Thickener (D): hydroxypropyl cellulose (product name Metholose SH15000, manufactured by Shin-Etsu Chemical Co., Ltd.)
Antifoaming agent: BYK-011 (product name, manufactured by Byk-Chemie (Germany), mixture of foam-breaking polymer and hydrophobic particles (non-silicone type))

### Example 1

As shown in Table 1, 40 parts by mass of the silver-based inorganic antibacterial agent, 1.8 parts by mass of the dispersant, 2.4 parts by mass of the discoloration inhibitor (A), 0.2 parts by mass of the thickener (C), and 0.1 parts by mass of the antifoaming agent were added to 55.5 parts by mass of water as a dispersion medium and stirred by means of a sand mill at 2000 rpm for 1 hour to give a silver-based inorganic antibacterial agent dispersion.

### Example 2

A silver-based inorganic antibacterial agent dispersion was prepared in the same manner as in Example 1 except that 1.0 parts by mass of the discoloration inhibitor (B) was used instead of the discoloration inhibitor (A), 57 parts by mass of water was used as a dispersant, and the antifoaming agent was not used.

### Example 3

A silver-based inorganic antibacterial agent dispersion was prepared in the same manner as in Example 1 except that 1.2 parts by mass of the discoloration inhibitor (A), 0.5 parts by mass of the discoloration inhibitor (B), and 56.2 parts by mass of water as a dispersant were used.

### Comparative Example 1

A silver-based inorganic antibacterial agent dispersion for a comparison example was prepared in the same manner as in Example 1 except that the discoloration inhibitor was not used.

### Comparative Example 2

A silver-based inorganic antibacterial agent dispersion for a comparison example was prepared in the same manner as in Example 1 except that the thickener was not used.

### Comparative Example 3

A silver-based inorganic antibacterial agent dispersion for a comparison example was prepared in the same manner as in Example 1 except that the dispersant was not used.

### Comparative Example 4

A silver-based inorganic antibacterial agent dispersion for a comparison example was prepared in the same manner as in Example 1 except that 0.02 parts by mass of the thickener (C) was used.

### Comparative Example 5

A silver-based inorganic antibacterial agent dispersion for a comparison example was prepared in the same manner as in Example 1 except that 0.2 parts by mass of the thickener (D) was used instead of the thickener (C).

### Comparative Example 6

An attempt was made to prepare a silver-based inorganic antibacterial agent dispersion in the same manner as in Example 1 except that 70 parts by mass of the silver-based inorganic antibacterial agent was used (here, the dispersion medium was used at 25.5 parts by mass so as to make a total of 100 parts by mass), but the viscosity was very high, and a dispersion having good dispersibility could not be obtained.

An attempt was made to prepare a silver-based inorganic antibacterial agent dispersion in the same manner as in Example 1 except that 5 parts by mass of the thickener was used (here, the dispersion medium was used at 50.7 parts by mass so as to make a total of 100 parts by mass), but the viscosity was very high, and a dispersion having good dispersibility could not be obtained.

TABLE 1

| | Antibacterial | Discoloration | | | Dispersion | Antifoaming |
|---|---|---|---|---|---|---|
| | agent | inhibitor | Thickener | Dispersant | medium | agent |
| Ex. 1 | 40 | (A) 2.4 | (C) 0.2 | 1.8 | 55.5 | 0.1 |
| Ex. 2 | 40 | (B) 1 | (C) 0.2 | 1.8 | 57 | None |
| Ex. 3 | 40 | (A) 1.2 | (C) 0.2 | 1.8 | 56.2 | 0.1 |
| | | (B) 0.5 | | | | |
| Comp. | 40 | None | (C) 0.2 | 1.8 | 57.9 | 0.1 |
| Ex. 1 | | | | | | |
| Comp. | 40 | (A) 2.4 | None | 1.8 | 55.7 | 0.1 |
| Ex. 2 | | | | | | |
| Comp. | 40 | (A) 2.4 | (C) 0.2 | None | 57.3 | 0.1 |
| Ex. 3 | | | | | | |
| Comp. | 40 | (A) 2.4 | (C) 0.02 | 1.8 | 55.68 | 0.1 |
| Ex. 4 | | | | | | |
| Comp. | 40 | (A) 2.4 | (D) 0.2 | 1.8 | 55.5 | 0.1 |
| Ex. 5 | | | | | | |

### Example 4

### Evaluation of Stability of Various Types of Silver-Based Inorganic Antibacterial Agent Dispersions

The silver-based inorganic antibacterial agent dispersions prepared in Examples 1 to 3 and Comparative Examples 1 to 5 were subjected to visual examination in terms of dispersibility, and the results are shown in Table 2. An evaluation of ‘Good’ was given when the dispersibility was good and precipitation did not occur, as ‘Some precipitate’ when precipitation occurred, and ‘Some aggregate’ when there were aggregates.

The silver-based inorganic antibacterial agent dispersions prepared in Examples 1 to 3 and Comparative Examples 1 to 5 were charged into 1 L polyethylene bottles and left to stand at 30° C. for 2 months. Subsequently, the height of the supernatant liquid (mm) was measured, and precipitation properties were evaluated. These results are also shown in Table 2.

TABLE 2

| | Results of | |
|---|---|---|
| | visual | Height of |
| Dispersion | examination | supernatant |
| Ex. 1 | Good | 2 mm |
| Ex. 2 | Good | 1 mm |
| Ex. 3 | Good | 1 mm |
| Comp. Ex. 1 | Good | 2 mm |
| Comp. Ex. 2 | Some | 70 mm |
| | precipitate | |
| Comp. Ex. 3 | Some | 25 mm |
| | aggregate | |
| Comp. Ex. 4 | Some | 67 mm |
| | precipitate | |
| Comp. Ex. 5 | Some | 72 mm |
| | precipitate | |

### Example 5

### Evaluation of Coating Prepared by Adding Silver-Based Inorganic Antibacterial Agent Dispersion to Paint

0.5 parts by mass of the silver-based inorganic antibacterial agent dispersion prepared in the Example was added to 99.5 parts by mass of a water-based UV paint (acrylic), and stirred well so as to form a dispersion. 5 g of this dispersion was placed on a 20 cm×10 cm piece of polypropylene film (OHP film) and made into a uniform coating using a bar coder (#60). This was dried at 50° C. for 10 minutes, and then irradiated with ultraviolet rays (80 W, 10 m/min, distance from light source: 10 cm, three passes) so as to cure the coating, thus giving a coating with added antibacterial agent (Coating 1).

Coatings 2 and 3 and Comparative coatings 1 to 5 were prepared by the same procedure as above using the silver-based inorganic antibacterial agent dispersions prepared in Examples 2 and 3 and Comparative Examples 1 to 5.

A comparative control coating was prepared by the same procedure but without using the silver-based inorganic antibacterial agent dispersion (Comparative control coating 1).

Coatings 1 to 3, Comparative coatings 1 to 5, and Comparative control coating 1 thus prepared were each cut to a size of 5.0 cm×5.0 cm, and evaluated in terms of state of coating, light discoloration resistance, and antibacterial power. These results are given in Table 3.

State of Coating

The state of Coatings 1 to 3, Comparative coatings 1 to 5, and Comparative control Coating 1 was visually examined in terms of the presence of aggregates, and an evaluation of ‘Good’ was given for those without aggregates and ‘Aggregated’ for those with aggregates.

Evaluation of Light Discoloration Resistance

The light discoloration resistance of Coatings 1 to 3, Comparative coatings 1 to 5, and Comparative control coating 1 was evaluated by exposing them in a sunshine weather meter (sunshine carbon arc lamp type JIS B 7753) for 200 hours and measuring the difference in color between that before and that after the light resistance test.

The color difference (ΔE) was determined from Equation [1] below using measurement values of color (L₁, a₁, b₁) before the light resistance test and color (L₂, a₂, b₂) after the test measured using a colorimeter (SZ-80 calorimeter, manufactured by Nippon Denshoku Industries Co., Ltd.).
ΔE=((L₁−L₂)²+(a₁−a₂)²+(b₁−b₂)²)^{1/2} [1]
Antibacterial Activity Test

The antibacterial activity of Coatings 1 to 3, Comparative coatings 1 to 5, and Comparative control coating 1 was evaluated in accordance with JIS Z2801.

E. coli was used as a test bacterium, and a bacterium solution was prepared to give a cell count of 2.5 to 10×10⁵ cells/mL in a solution of a standard broth culture medium in sterile water at a dilution of 1/500. 0.4 mL of the bacterium solution was dropped on the surface of the sample, the surface was then covered with a 4.0 cm×4.0 cm piece polyethylene film so as to make uniform contact with the surface, and the sample was stored at a temperature of 35° C. and a humidity of 95% RH for 24 hours. 0 hours (theoretical added cell count) and 24 hours after starting the storage, surviving cells on the sample were washed with 10 mL of a cell count medium (SCDLP liquid medium), and the washings were subjected to a viable cell count by the pour-plate culture method (37° C. 2 days) using a standard agar medium, and a viable cell count per sample was obtained.

TABLE 3

| | | State of | | Viable cell |
|---|---|---|---|---|
| Coating | Dispersion | coating | ΔE | count |
| Comparative control | None | Good | 1.2 | 1.7 × 10 ⁷ |
| coating 1 | | | | |
| Coating 1 | Ex. 1 | Good | 2.5 | <10 |
| Coating 2 | Ex. 2 | Good | 2.9 | <10 |
| Coating 3 | Ex. 3 | Good | 1.1 | <10 |
| Comparative coating 1 | Comp. Ex. 1 | Good | 11 | <10 |
| Comparative coating 2 | Comp. Ex. 2 | Good | 2.5 | <10 |
| Comparative coating 3 | Comp. Ex. 3 | Aggregated | 2.4 | 2.3 × 10³ |
| Comparative coating 4 | Comp. Ex. 4 | Good | 2.4 | <10 |
| Comparative coating 5 | Comp. Ex. 5 | Good | 2.6 | <10 |

As is clear from the color difference of Coating 3 in Table 3, a coating employing a plurality of discoloration inhibitors gave better results than one employing a single discoloration inhibitor.

### Example 6

### Evaluation of Cotton Cloth Processed Using Silver-Based Inorganic Antibacterial Agent Dispersion

A suspension was prepared by adding 10 parts by mass of the silver-based inorganic antibacterial agent dispersion (containing 4 parts by mass of the antibacterial agent) prepared in Examples 1 and 6.7 parts by mass of an acrylic binder (KB-4900, solids content 45%, manufactured by Toagosei Co., Ltd.) to 100 parts by mass of pure water. 100% cotton cloth was dipped in the suspension (cloth weight 100 g/m²), picked up at a squeeze rate of 70%, and dried at 150° C. to give Test cloth 1.

The silver-based inorganic antibacterial agent dispersions prepared in Examples 2 and 3 and Comparative Example 1 to 5 were subjected to the same procedure to give Test cloths 2 and 3 and Comparative cloths 1 to 5.

Evaluation of Feel

The feel and color of Test cloths 1 to 3 and Comparative cloths 1 to 5 were evaluated by hand and by eye.

An evaluation of ‘Good’ was given for those that had no discoloration and that did not have a rough feel, ‘Discolored’ for those exhibiting discoloration, and ‘Aggregated’ for those exhibiting a rough feel when touched or aggregates when examined visually. These results are given in Table 4.

Washing Test

Test cloths 1 to 3 and Comparative cloths 1 to 5 were washed 10 times, and the antibacterial action thereof was evaluated.

The results are given in Table 4. Evaluation of the antibacterial action was carried out by a quantitative analysis test in accordance with JIS L 1902⁻¹⁹⁹⁸, and the test was carried out using Staphylococcus aureus. When the bacteriostatic activity value was 2.2 or greater, it was evaluated as having antibacterial action. Washing was carried out in accordance with JIS L 0217, 103 (JAFFET standard detergent used).

TABLE 4

| Sample | Dispersion | Feel | Antibacterial action |
|---|---|---|---|
| Control cloth | None | — | None |
| Test cloth 1 | Ex. 1 | Good | Yes |
| Test cloth 2 | Ex. 2 | Good | Yes |
| Test cloth 3 | Ex. 3 | Good | Yes |
| Comparative cloth 1 | Comp. Ex. 1 | Discolored | Yes |
| Comparative cloth 2 | Comp. Ex. 2 | Good | Yes |
| Comparative cloth 3 | Comp. Ex. 3 | Aggregated | Yes |
| Comparative cloth 4 | Comp. Ex. 4 | Good | Yes |
| Comparative cloth 5 | Comp. Ex. 5 | Good | Yes |

### Example 7

### Evaluation of Cotton Cloth Processed Using Silver-Based Inorganic Antibacterial Agent Dispersion

A suspension was prepared by adding 1 part by mass of the silver-based inorganic antibacterial agent dispersion prepared in Example 1 (containing 0.4 parts by mass of the antibacterial agent) and 6.7 parts by mass of an acrylic binder (KB-4900, solids content 45%, manufactured by Toagosei Co., Ltd.) to 100 parts by mass of pure water. 100% cotton cloth was dipped in the suspension (cloth weight 100 g/m²), picked up at a squeeze rate of 70%, and dried at 150° C. to give Test cloth 4.

The silver-based inorganic antibacterial agent dispersions prepared in Examples 2 and 3 and Comparative Examples 1 to 5 were subjected to the same procedure to give Test cloths 5 and 6 and Comparative cloths 6 to 10.

The feel and antibacterial action of Test cloths 4 to 6 and Comparative cloths 6 to 10 were evaluated in the same manner as in Example 6, and the results are given in Table 5.

TABLE 5

| Sample | Dispersion | Feel | Antibacterial action |
|---|---|---|---|
| Control cloth | None | — | None |
| Test cloth 4 | Ex. 1 | Good | Yes |
| Test cloth 5 | Ex. 2 | Good | Yes |
| Test cloth 6 | Ex. 3 | Good | Yes |
| Comparative cloth 6 | Comp. Ex. 1 | Discolored | Yes |
| Comparative cloth 7 | Comp. Ex. 2 | Good | Yes |
| Comparative cloth 8 | Comp. Ex. 3 | Good | None |
| Comparative cloth 9 | Comp. Ex. 4 | Good | Yes |
| Comparative cloth 10 | Comp. Ex. 5 | Good | Yes |

The results for Example 6 and Example 7 are summarized in Table 6.

Stability was described with respect to whether or not a dispersed state was maintained when the silver-based inorganic antibacterial agent dispersion, etc. was stored for a long period of time. This was evaluated as follows.
A: Excellent
B: Good
C: Poor

An OHP film was coated with a water-based ultraviolet curing paint containing the silver-based inorganic antibacterial agent dispersion, etc., cured, and evaluated in terms of the state of the coating, color resistance, and antibacterial action, the evaluation being given as follows.
A: Excellent
B: Good
C: Poor

Cotton cloth was dipped in a suspension containing the silver-based inorganic antibacterial agent dispersion, etc. and evaluated in terms of feel and antibacterial action, the evaluation being given as follows.
A: Excellent
B: Good

C: Poor

TABLE 6

| | | | | | | Overall |
|---|---|---|---|---|---|---|
| | | Disper- | | Coated | | evalu- |
| Sample | | sion | Stability | sheet | Fiber | ation |
| Coating 1 | Test cloths | Ex. 1 | B | B | B | B |
| | 1, 4 | | | | | |
| Coating 2 | Test cloths | Ex. 2 | B | B | B | B |
| | 2, 5 | | | | | |
| Coating 3 | Test cloths | Ex. 3 | B | A | B | A |
| | 3, 6 | | | | | |
| Comparative | Comparative | Comp. | B | C | C | C |
| coating 1 | cloths 1, 6 | Ex. 1 | | | | |
| Comparative | Comparative | Comp. | C | B | B | C |
| coating 2 | cloths 2, 7 | Ex. 2 | | | | |
| Comparative | Comparative | Comp. | C | C | C | C |
| coating 3 | cloths 3, 8 | Ex. 3 | | | | |
| Comparative | Comparative | Comp. | C | B | B | C |
| coating 4 | cloths 4, 9 | Ex. 4 | | | | |
| Comparative | Comparative | Comp. | C | B | B | C |
| coating 5 | cloths 5, 10 | Ex. 5 | | | | |

It can be seen that the silver-based inorganic antibacterial agent dispersions of Examples 1 to 3 were superior to those of Comparative Examples in all respects, including stability of dispersion, and application to coated sheet and fiber. On the other hand, the dispersions of the Comparative Examples were poor in some respects.

### Example 8

### Evaluation of Color of Black Cotton Cloth Processed Using Silver-Based Inorganic Antibacterial Agent Dispersion

A processing liquid was prepared by adding 2.5 parts by mass of the silver-based inorganic antibacterial agent dispersion prepared in Example 1 (containing 1 part by mass of the antibacterial agent), 3.7 parts by mass of an acrylic binder (KB-4900, solids content 45%, manufactured by Toagosei Co., Ltd.), and 0.5 parts by mass of a silica sol (ST-C, Nissan Chemical Industries Ltd., SiO₂ concentration about 20 wt %) to 100 parts by mass of pure water. 100% cotton black cloth (L value =16.2, cloth weight 100 g/m²) was dipped in the processing liquid, picked up at a squeeze rate of 100%, and dried at 150° C. to give Test cloth 7.

A processing liquid was prepared by adding 2.5 parts by mass of the silver-based inorganic antibacterial agent dispersion prepared in Example 1 (containing 1 part by mass of the antibacterial agent), 3.7 parts by mass of an acrylic binder (KB-4900, solids content 45%, manufactured by Toagosei Co., Ltd.), and 0.5 parts by mass of an alumina sol (Alumina sol 520, Nissan Chemical Industries Ltd., Al₂O₃ concentration about 25%) to 100 parts by mass of pure water. 100% cotton black cloth (L value=16.2, cloth weight 100 g/m²) was dipped in the processing liquid, picked up at a squeeze rate of 100%, and dried at 150° C. to give Test cloth 8.

A processing liquid was prepared by adding 2.5 parts by mass of the silver-based inorganic antibacterial agent dispersion prepared in Example 1 (containing 1 part by mass of the antibacterial agent) and 3.7 parts by mass of an acrylic binder (KB-4900, solids content 45%, manufactured by Toagosei Co., Ltd.) to 100 parts by mass of pure water. 100% cotton black cloth (L value=16.2, cloth weight 100 g/m²) was dipped in the processing liquid, picked up at a squeeze rate of 100%, and dried at 150° C. to give Comparative cloth 11.

The color of these Test cloths was examined by eye. The color was further measured in terms of color (L, a, b) using a calorimeter (SZ-Σ80 colorimeter, manufactured by Nippon Denshoku Industries Co., Ltd.). By comparing the color before and after processing with the dispersion, a color difference ΔE was obtained. The results are given in Table 7.

TABLE 7

| | | Color of cloth | | | Color of cloth | | | |
|---|---|---|---|---|---|---|---|---|
| | | before | | | after | | | Color |
| | | processing | | | processing | | | difference |
| | Color | L | a | b | L | a | b | ΔE |
| Test cloth 7 | Attractive black | 16.0 | 0.4 | −0.3 | 15.8 | 0.4 | −0.6 | 0.4 |
| | similar to | | | | | | | |
| | unprocessed cloth | | | | | | | |
| Test cloth 8 | Attractive black | 16.0 | 0.4 | −0.3 | 16.1 | 0.3 | −0.2 | 0.2 |
| | similar to | | | | | | | |
| | unprocessed cloth | | | | | | | |
| Comparative cloth | White patches | 16.0 | 0.4 | −0.3 | 19.2 | 0.2 | −1.6 | 3.5 |
| 11 | | | | | | | | |

### INDUSTRIAL APPLICABILITY

It is possible to provide the silver-based inorganic antibacterial agent dispersion of the present invention, which is stable and causes little precipitation even during long term storage. Furthermore, since the silver-based inorganic antibacterial agent dispersion of the present invention has good dispersibility during addition to a paint and during processing of cloth, etc., it has excellent processing properties. From this, it is possible to produce excellent antibacterial products having uniform antibacterial activity.

1. A silver-based inorganic antibacterial agent dispersion comprising a silver-based inorganic antibacterial agent, a discoloration inhibitor, a thickener, a dispersant, and a dispersion medium, wherein the silver-based inorganic antibacterial agent is 5 to 60 parts by mass relative to 100 parts by mass of the silver-based inorganic antibacterial agent dispersion, and the thickener is 0.1 to 10 parts by mass relative to 100 parts by mass of the silver-based inorganic antibacterial agent.

2. (canceled)

3. A silver-based inorganic antibacterial agent dispersion formed by further adding a fine particulate compound having an average particle size of 1 to 100 nm to the silver-based inorganic antibacterial agent dispersion according to claim 1.

4. (canceled)

5. The silver-based inorganic antibacterial agent dispersion according to claim 1, wherein the discoloration inhibitor is an imidazole series compound and/or a benzotriazole series compound.

6. The silver-based inorganic antibacterial agent dispersion according to claim 1, wherein the thickener is a polysaccharide and/or a cellulose-based thickener.

7. The silver-based inorganic antibacterial agent dispersion according to claim 1, wherein the dispersant is an anionic surfactant and/or a nonionic surfactant.

8. The silver-based inorganic antibacterial agent dispersion according to claim 1, wherein it further comprises a binder resin.

9. An antibacterial-processed product processed using the silver-based inorganic antibacterial agent dispersion according to claim 1.
